(19) United States
(12) Patent Application Publication
(10) Pub. No.: US 2021/0363106 A1
(43) Pub. Date: Nov. 25, 2021

(54) PROCESS FOR PRODUCING SUBSTITUTED 2-ALLYLANILINES AND SUBSTITUTED 4-AMINOINDANES

(71) Applicant: BAYER AKTIENGESELLSCHAFT, Leverkusen (DE)

(72) Inventors: Florian ERVER, Wiesbaden (DE); Mark James FORD, Niedernhausen (DE)

(21) Appl. No.: 17/056,986

(22) PCT Filed: May 21, 2019
(86) PCT No.: PCT/EP2019/063063

§ 371 Date: Nov. 19, 2020

(30) Foreign Application Priority Data
May 23, 2018 (EP) 18173856.8
May 23, 2018 (EP) 18173859.2

(51) Int. Cl. C07D 213/82; C07C 209/10; C07C 209/68; C07C 209/54

(52) U.S. Cl. CPC C07D 213/82; C07C 209/10; C07C 2602/08; C07C 209/54; C07C 2601/16; C07C 209/68

(57) ABSTRACT

The present invention primarily relates to a process for producing certain substituted 2-allylanilines of the hereinbelow-defined formula (I) and their use in a process for producing substituted 4-aminoindane derivatives of the hereinbelow-defined formula (V). The present invention further relates to a process for producing fungicidal indanyl carboxamides. In particular, the present invention relates to a process for producing 2-(difluoromethyl)-N-(1,1-dimethyl-3-propyl-2,3-dihydro-1H-inden-4-yl)nicotinamide and/or 3-(difluoromethyl)-N-[(R)-2,3-dihydro-1,1,3-trimethyl-1H-inden-4-yl]-1-methylpyrazole-4-carboxamide.

The present invention primarily relates to a process for producing certain substituted 2-allylanilines of the hereinbelow-defined formula (I) and their use in a process for producing substituted 4-aminoindane derivatives of the hereinbelow-defined formula (V). The present invention further relates to a process for producing fungicidal indanyl carboxamides. In particular, the present invention relates to a process for producing 2-(difluoromethyl)-N-(1,1-dimethyl-3-propyl-2,3-dihydro-1H-inden-4-yl)nicotinamide and/or 3-(difluoromethyl)-N-[(R)-2,3-dihydro-1,1,3-trimethyl-1H-inden-4-yl]-1-methylpyrazole-4-carboxamide.

### Substituted 2-allylanilines of the General Formula (I)

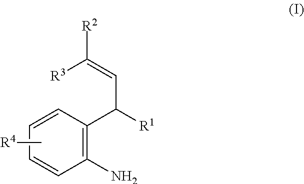

(i.e. compounds of the formula (I)) are valuable intermediates in various industrial fields, in particular for producing biologically active substances which can be employed in the pharmaceutical and/or agrochemical sector. In particular, they are valuable precursors in the production of substituted 4-aminoindane derivatives of the general formula (V)

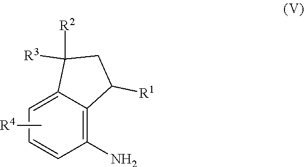

(i.e. compounds of the formula (V)), which again are valuable intermediates in the production of fungicidal indanyl carboxamides. Such fungicidal indanyl carboxamides and methods for their production are known e.g. from WO 1992/12970; WO 2012/065947; J. Org. Chem. 1995, 60, 1626-1631; WO 2012/084812; WO2010/109301; EP 0256503; JP-A 1117864; J. Pesticide Sci. 1993, 18, 245-251; EP 2940001; EP 0654464; WO 2017/178868; WO 2014/095675, WO 2015/197530 and WO 2017/133981.

Very generally, fungicidal indanyl carboxamides can be synthesized via the coupling of substituted 4-aminoindane derivatives with an activated heterocyclic acid counterpart by linking the primary amino group of the former with the carboxyl group of the latter (coupling reaction). Therefore, substituted 4-aminoindane derivatives, but also the activated heterocyclic acids that shall be linked to the substituted 4-aminoindane derivatives, are important intermediates in the synthesis of fungicidal indanyl carboxamides.

In particular, WO 2017/133981 discloses a process for the synthesis of substituted 4-aminoindane derivatives via substituted 2-allylanilines: It is described that appropriately substituted 2-aminobenzonitriles shall be used as starting material in reaction with appropriate Grignard reagents to prepare first anilines having hydroxyalkyl side chains. Secondly, such are dehydrated, isomerized and cyclized to their corresponding 4-aminoindane derivatives mediated by sulphonic acids.

While this prior art process for producing substituted 4-aminoindane derivatives allows the production of the desired compounds in some cases in an acceptable yield, it does still have disadvantages:

For example, in view of the 2-aminobenzonitriles as starting material, WO 2017/133981 discloses that such are only in some cases commercially available, i.e. they are rarely available on technical scale. Concluding, already WO 2017/133981 itself indicates that 2-aminobenzonitriles are not obtainable on a reliable and regular basis which is a disadvantage for the large scale production of substituted 4-aminoindane derivatives and the downstream products produced thereof.

Other disadvantages of the process according to WO 2017/133981 are the intermediates produced during the synthesis: Starting from the alcohols, reaction with sulphonic acid leads firstly to a mixture of several 2-allylaniline isomers, which may interconvert by isomerization. From this mixture, only the substituted 2-allylanilines of the formula (VIb) are suitable as cyclization precursors and can cyclize irreversibly to the desired substituted 4-aminoindane derivatives.

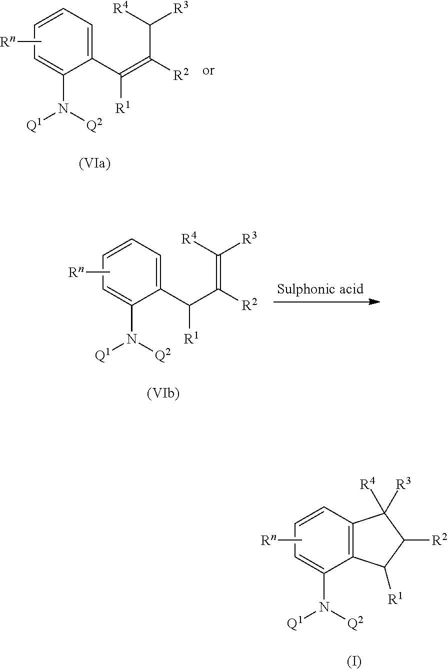

Concluding, the process according to WO 2017/133981 does not allow a selective synthesis of the relevant substituted 2-allylanilines in a straightforward fashion which makes it is less efficient and energy-wasting.

Condensation reactions of anilines with allylhalides, which would not need an application of expensive transition metals, are in principle known from Wolf and Ramin. They reported condensations of aniline with 3-chlorobut-1-ene and also 3-methyl-3-chlorobut-1-ene, accessed from the corresponding alcohols by dehydroxychlorination, in the presence of sodium carbonate in ethanol at 78° C. to obtain N-(1-methylallyl)aniline in 47% yield and N-(1,1-dimethylallyl)aniline in 24% yield, respectively (V. Wolf, D. Ramin, Eur. J. Org. Chem. 1959, p 47-60). Edmundson and Moran reported the condensation of aniline with 1-chloro-3-methylbut-2-ene, which was carried out at 100° C. without solvent and led to a product mixture, which was extensively purified by multiple distillations and a final chromatography in order to obtain N-(3-methylbut-2enyl)aniline in 25% yield and in pure form (R. S. Edmundson, T. A. Moran, J. Chem. Soc. 1980, p 1009-1012). In a review of Jolidon and Hansen the condensation of 2-substituted anilines with 3-chlorobut-1-ene has been described. In the case of allylation of unsubstituted aniline they were able to obtain N-(1-methylallyl)aniline in 11% yield after separation from the regioisomeric side-product N-(3-methylallyl)aniline (S. Jolidon, H.-J. Hansen, Helv. Chim. Act. 1977, p 978-1031). The condensation reaction led to the mixture of regioisomers in 65% yield and a regioisomeric ratio of 75:25 in favor of N-(1-methylallyl)aniline. Anilines with substituents like methyl (Me) or isopropyl (ⁱPr) in the 2-position led to even lower overall yields (Me: 56%, ⁱPr: 27%) with varying regioselectivities, which did not exceed 75%. Jolidon and Hansen also described the condensation of aniline and 1-bromo-3-methyl-but-2-ene mediated by anhydrous sodium carbonate in ethanol at 78° C., which led to N-(3-methylbut-2enyl)aniline in 14% yield after two distillations of the initial product mixture, which contained 30% of N-(3-methylbut-2enyl)aniline and 23% of the doubly allylated product N,N-Bis(3-methylbut-2enyl)aniline. This result was validated by WO 2008/15240, in which the same condensation is reported, using anhydrous potassium carbonate in tetrahydrofuran under ambient conditions, leading to N-(3-methylbut-2enyl)aniline in 23% yield after column chromatography. However, a more efficient approach was reported by Alcantara. In this procedure the condensation of aniline with 1-bromo-3-methyl-but-2-ene was mediated by KF-Celite in anhydrous acetonitrile at 82° C. The product, N-(3-methylbut-2enyl)aniline, was accompanied by 10% of N,N-Bis(3-methylbut-2enyl)aniline and was obtained in 83% yield after chromatography (Alcantara et al., Org. Lett. 2007, p 2661-2664). Another allylation of unsubstituted aniline with an unsymmetrical allylbromide, namely (E)-1-bromobut-2-ene, was published earlier by Barluenga. In this context, the terms “unsymmetrical” and “symmetrical” respectively refer to the molecular symmetry of the allyl system. The allyl functionality consists of three carbon atoms, which are connected with each other via sigma bonds and having two n-electrons delocalized over the three atoms with equal distribution of electron density. In a case where the central carbon atom is part of a mirror plane, which is orthogonal to the sigma bonds, “symmetrical” means that both sides of the mirror plane are identical, while “unsymmetrical” means they are not. The condensation was simply carried out in water at 50-65° C. and led to the desired product N-[(E)-but-2-enyl]aniline in 86% yield (J. Barluenga et al., J. Chem. Res. 1989, p 1524-1552).

Finally, not only condensations with allylhalides but also dehydrative condensations with allylalcohols are known. For example, Kubota described a protocol where rarely available bis(2,2,2-trifluoroethoxy)triphenylphosphorane was utilized in stoichiometric amounts to mediate the condensation of (E)-but-2-en-1-ol and but-3-en-2-ol with aniline to receive N-[(E)-but-2-enyl]aniline and N-(1-methylallyl)aniline in 64% and 66% yield, respectively (Kubota et al., J. Org. Chem. 1980, p 5052-5057). A later publication of Kaneda also dealt with the condensation of unsubstituted aniline and but-3-en-2-ol mediated by catalytic amounts of a more readily available proton-exchanged montmorillonite catalyst at 150° C. in n-heptane, which led to N-[(E)-but-2-enyl]aniline in 63% yield (Kaneda et al., Org. Lett. 2006, p 4617-4620). In summary the most efficient allylations of aniline were described by Alcantara and Barluenga using unsymmetrical allylbromides, which led to yields of substituted N-allylanilines of >80%. However, methods for the production of trisubstituted unsymmetrical N-allylanilines could not be found so far.

Some examples for Ammonium-Claisen rearrangements (ACRs) of N-allylanilines with unsymmetrical substitution patterns on the allylic moiety are known. A major part of pioneering work is described again in the review of Jolidon and Hansen: Depending on the substituent (R═OMe, Cl, ⁱPr) in the 4-position of the N-(1,1-dimethyl-allyl)aniline, the ACR was complete after 3-8 hours in presence of a mediator like 0.2 M sulfuric acid or a mixture of trifluoroacetic acid/water/1,4-dioxane. Low to moderate yields of 20-70% were obtained (S. Jolidon, H.-J. Hansen, Helv. Chim. Act. 1977, p 978-1031). Building on these results Ward et al. described a method of converting also N-(1,1-disubstituted-allyl)anilines into the corresponding ACR products using catalytic amounts of 4-toluenesulfonic acid in acetonitrile/water in a ratio of 10:1 at 65° C. achieving yields of 52-67% (A. D. Ward et al., Synthesis 2001, p 621-625). A similar yield (55%) was obtained by Brucelle and Renaud after mediating the ACR of N-(3-methyl-allyl)aniline in xylenes in presence of stoichiometric amounts of trifluoroboroetherate at 145° C. (F. Brucelle & P. Renaud, J. Org. Chem. 2013, p 6245-6252).

Chemical syntheses of substituted 4-aminoindane derivatives have been described e.g. in WO 2010/109301, WO 2014/103811 and U.S. Pat. No. 5,521,317. However, the described processes only allow the preparation of substituted 4-aminoindanes with very limited substitution patterns. For instance, the methods described in WO 2010/109301 and in WO 2014/103811 only allow the synthesis of an 1,1,3-trimethyl-4-aminoindane derivative starting from aniline by condensation with acetone and exploit the rearrangement reaction described in EP 0654464 and U.S. Pat. No. 5,521,317.

EP 0654464 discloses that 4-aminoindane derivatives can be obtained in four steps comprising i) condensation between a dihydroquinoline and a carboxylic acid derivative; ii) catalytic hydrogenation to provide the corresponding tetrahydroquinoline; iii) addition of a strong acid to obtain the corresponding 4-aminoindane derivative; and iv) hydrolysis of the amide bond.

In contrast, WO 2017/178868 describes the same process as EP 0654464 but discloses that by inverting the steps of ii) hydrogenation and i) condensation, it is possible to prepare 4-aminoindane derivatives and the corresponding amides in a simpler and more cost-effective way.

EP 2940001 discloses a method for producing a purified amine compound (i.e. a substituted 4-aminoindane derivative). According to EP 2940001, it is crucial to obtain a highly pure amine compound for the industrial production of a highly pure N-indanyl carboxamide compound. To obtain first a crude amine compound, EP 2940001 discloses that a dihydroquinoline derivative can be hydrogenated to yield an intermediary compound which is then reacted with an acid. The resultant reaction mixture is mixed with water and neutralized with an alkaline solution, extracted with an organic solvent insoluble in water, to yield the crude amine compound. In order to obtain the final product of the formula (1) from this crude amine compound, EP 2940001 describes a process of four steps (A) to (D), comprising (A) reacting the crude amine compound with a hydrogen halide and further (B) separation, (C) precipitating and (D) isolation of the hydrogen halide salt of the amine compound produced in step (A) and finally reacting this salt with a base.

A further possibility to prepare 4-aminoindane derivatives is described in WO 2013/167545 and WO 2013/167549. The synthesis is based on a Buchwald-Hartwig amination and thus enables a general synthetic route to substituted 4-aminoindanes. Disadvantages of this method are firstly the cost-intensive use of transition metal catalysts and secondly the problematic synthesis of the corresponding halo-substituted indane precursors. Furthermore, the amino function cannot be introduced directly by free NH₃, but rather requires the use of cost-intensive, protected ammonia derivatives.

Indanes without an amino function on the aromatic ring can be prepared by methods established in classical organic chemistry by Friedel-Crafts cyclizations. To this end, aromatic compounds having hydroxyalkyl or alkene side chains are converted to the corresponding indanes by addition of Brønsted acids such as HCl, HBr, HF, H₂SO₄, H₃PO₄, KHSO₄, AcOH, p-toluenesulfonic acid, polyphosphoric acid or of Lewis acids such as AlCl₃, BF₃, AgOTf.

However, it has been shown that, with the exception of polyphosphoric acid, none of the reagents mentioned can be used to prepare 4-aminoindane derivatives by cyclization (J. S. Pizey (Ed.), “Synthetic Reagents 6” Wiley-VCH: New York 1985, 156-414).

In line with this, WO 2015/197530 discloses a preparation example in which the reaction of such aromatic compounds having hydroxyalkyl side chains with polyphosphoric acid at a temperature of 80° C. led successfully to the formation of substituted 4-aminoindane derivatives.

Surprisingly, WO 2017/133981 discloses that substituted 4-aminoindane derivatives can be prepared from aromatic compounds having hydroxyalkyl side chains which are converted to the corresponding 4-aminoindane derivatives by addition of sulfonic acids. In detail, WO 2017/133981 discloses the synthesis of substituted 4-aminoindane derivatives via utilizing sulfonic acids for the initial dehydration of the 2-(hydroxyalkyl)-anilines and subsequent isomerization of their immediate corresponding 2-(alkenyl)-anilines towards their 4-aminoindane cyclization precursor before final and irreversible cycloisomerization towards the target compounds.

While this prior art process for producing substituted indanylamines allows the production of the desired compounds in some cases in an acceptable yield, it does have disadvantages: As described, the reaction can be performed particularly well in the presence of either methanesulfonic acid (MsOH) or, most preferably, with trifluoromethanesulfonic acid (TfOH) as cyclization mediator. While MsOH is a readily available bulk chemical, TfOH displays limited availability and is consequently highly expensive. Even though the majority of the acid being used can in principle be recycled, at least one equivalent forms the respective 4-aminoindane trifluoromethylsulfonate salt as an immediate product. Said equivalent and potential TfOH residues on the salt cannot be recovered via distillation and have to be neutralized by a base. The costs for raw-material consumption and wastewater treatment add up significantly to the overall process costs. This issue is inferior for the case, when MsOH is being used, due to significantly lower raw-material costs and the good biodegradability of this acid to carbon dioxide and sulfate. However, in WO 2017/133981 is reported that the application of MsOH only led to moderate yields, e. g. 52% yield by HPLC, which is mainly due to the formation of isomeric olefins that do not cyclize to the desired product.

The condensation of the allyl halide A with the aniline B leads to a mixture of N-allylated compounds C1 and C2. This mixture isomerizes under Aza-Claisen-Rearrangement (ACR) conditions to two different 2-allylamines D1 and D2. Due to very similar physical properties D1 cannot be separated easily from C2 or D2. Subjecting this mixture to cyclization results in different aminoindane derivatives E1 and E2.

Summarizing, the process according to WO 2017/133981 uses either a cyclization mediator which is highly expensive and which is difficult to recycle but which leads to acceptable yields or uses a cyclization mediator which is less expensive and exhibits a good biodegradability but instead leads to lower yields.

Moreover, WO 2017/133981 discloses that when certain acids other than TfOH, MsOH or polyphosphoric acid are used, no yield is obtained with this process, especially no yield was generated when sulfuric acid was used as cyclization mediator.

Another disadvantage is the limited availability of the aforementioned 2-(hydroxyalkyl)-aniline substrates (i.e. 4-aminoindane cyclization precursors), which can be synthesized from their corresponding 2-aminobenzonitriles. According to WO 2017/133981, the latter are only in some cases commercially available.

Therefore, not only the most preferred mediator but also the substrates of the described process display limited availability, resulting in high process costs.

To overcome the above-mentioned disadvantages in view of the process described in WO 2017/133981, it is accordingly an object of the present invention to find a new process for the production of substituted 2-allylanilines in which a starting material is used that is obtainable on a reliable and regular basis. This would overcome high production costs and enable a large scale production of downstream products such as substituted 4-aminoindane derivatives and consequently fungicidal indanyl carboxamides.

Furthermore in view of WO 2017/133981, it is accordingly another object of the present invention to find a new process for the selective production of substituted 2-allylanilines which allows their synthesis in a straightforward fashion avoiding unnecessary isomerization of intermediates during the synthesis. With this, the process would be more efficient and more energy-saving. In particular, such a process would exhibit an improved space-time-yield.

With regard to the disadvantages outlined above, there is a demand for a simplified method that can be carried out industrially and economically for the general preparation of substituted 4-aminoindane derivatives. The substituted 4-aminoindane derivatives obtainable by this desired method should preferably in this case be obtained in high yield and high purity. In particular, the desired method should enable the desired target compounds to be obtained without the need for complex purification methods.

The hereinbelow-described processes according to the invention achieve these objects.

The process according to the invention affords the production of substituted 2-allylanilines using a starting material which is obtainable on a reliable and regular basis.

The process according to the invention affords the selective production of substituted 2-allylanilines allowing their synthesis in a straightforward fashion and thereby avoiding unnecessary isomerization of intermediates during the synthesis. This means, during the process according to the invention, fewer undesired secondary components are formed so that the process according to the invention is more efficient and more energy-saving.

The process according to the invention avoids elimination of the substituted allylalcohols to the corresponding diene and also the formation of regioisomers during the activation of the allylalcohol. “Activation” in this context means transformation of the hydroxyl group of allylalcohols into a suitable leaving group X.

The process according to the invention allows the activated allylalcohols and their potential regioisomers to be condensated to the substituted anilines in high regio- and chemoselectivity, so that the N-allylated intermediates lead to the desired substitution patterns of the substituted 2-allylanilines after the ACR.

Another advantage of the process according to the invention is the optimization of the ratio of generated regioisomers of the N-allylated intermediates in favor of the one, which is finally rearranged to the desired substituted 2-allylanilines.

The process according to the invention allows the separation of generated regioisomers of the N-allylated intermediates in a cost-effective manner. In particular, another advantage of the process according to the invention is the simplicity of the separation of the undesired regioisomers of the generated N-allylated intermediates which is achieved by simply increasing the temperature after the completed rearrangement reaction.

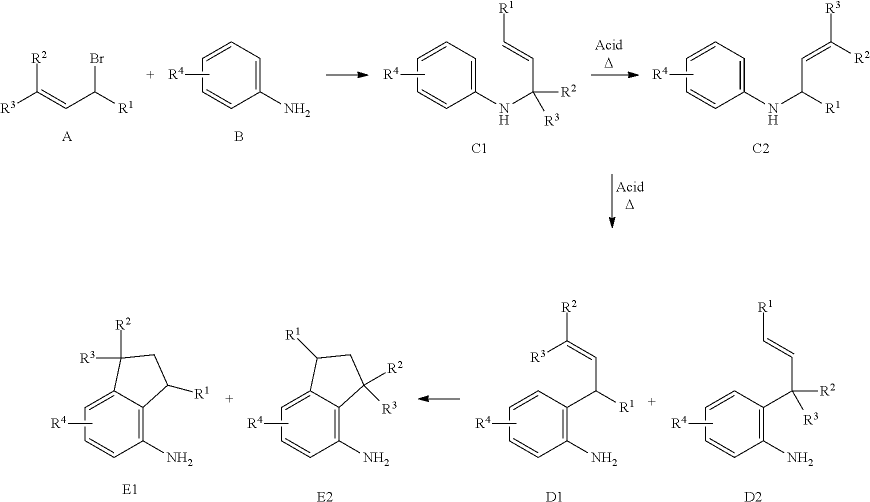

The process according to the invention avoids the isolation of intermediary compounds such as the activated allylalcohol or the N-allylated intermediates which maximizes the overall space-time-yield of the desired substituted 2-allylanilines. In particular, the process according to the invention can be conducted as a telescoping synthesis, i.e. it is workable as a sequential one-pot synthesis with reagents added to a reactor one at a time, wherein minimal work-up procedures are performed during the process. Minimal work-up procedures are e.g. separation and/or washing steps. In particular, during the process according to the invention the isolation of the activated allylalcohol can be avoided and instead the activated allylalcohol can be condensated directly with the substituted aniline.

The process according to the invention allows the production of substituted 4-aminoindane derivatives in a cost-efficient manner and in higher yields.

The process according to the invention allows a selective cycloisomerization of readily available substituted 2-allylanilines, instead of the dehydrative cyclization of the 2-(hydroxyalkyl)-aniline substrates as described in WO 2017/133981.

Furthermore, the process for production of substituted 4-aminoindane derivatives according to the invention allows the use of recyclable mediators during their synthesis. In particular, the process according to the invention allows the use of recyclable acids during the synthesis of said substituted 4-aminoindane derivatives. Consequently, the production of huge amounts of waste is prevented by the process according to the invention.

The present invention provides a process for the production of substituted 4-aminoindane derivatives via 2-allylanilines in high yields which is very well suitable for large scale production. The present invention provides a process for the preparation of a compound of the formula (V)

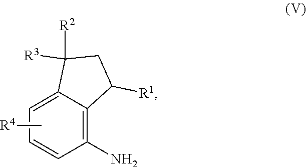

in which
R¹ represents (C₁-C₄)alkyl;
R² represents hydrogen or (C₁-C₈)alkyl;
R³ represents hydrogen or (C₁-C₈)alkyl, provided that R² and R³ are not hydrogen at the same time;
R⁴ represents hydrogen, halogen, (C₁-C₄)alkyl or (C₁-C₄)haloalkyl, characterized in that a compound of the Formula (I)

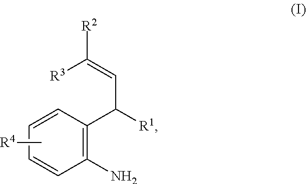

wherein the definitions of the substituents R¹, R², R³ and R⁴ listed in the formula (I) are the same as in the formula (V) is reacted with aqueous sulfuric acid or anhydrous hydrogen fluoride (HF).

Furthermore the present invention provides a process for the preparation of a compound of the formula (I)

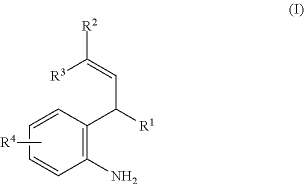

in which
R¹ represents (C₁-C₄)alkyl;
R² represents hydrogen or (C₁-C₈)alkyl;
R³ represents hydrogen or (C₁-C₈)alkyl, provided that R² and R³ are not hydrogen at the same time;
R⁴ represents hydrogen, halogen, (C₁-C₄)alkyl or (C₁-C₄)haloalkyl;
comprising the steps (b) and (c),
wherein in a step (b) a compound of the formula (III)

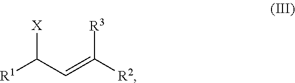

in which X represents a halogen or O—SO₂R wherein R is a methyl, phenyl or tolyl group and wherein the definitions of the substituents R¹, R² and R³ listed in the formula (III) are the same as in the formula (I) is reacted with a compound of the formula (IIIa)

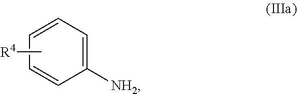

in which the definition of the substituent R⁴ listed in the formula (IIIa) is the same as in the formula (I), to obtain a compound of the formula (IV)

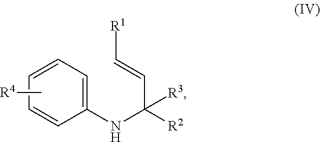

in which the definitions of the substituents R¹, R², R³ and R⁴ listed in the formula (IV) are the same as in the formula (I);
and wherein in a step (c) the compound of the formula (IV), wherein the definitions of the substituents R¹, R², R³ and R⁴ listed in the formula (IV) are the same as in the formula (I), is rearranged in the presence of an acid to obtain the compound of the formula (I).

The present invention furthermore provides a process for the preparation of a compound of the formula (I)

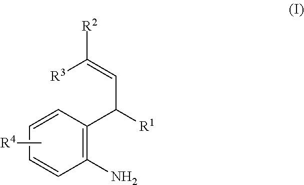

in which
R¹ represents (C₁-C₄)alkyl;
R² represents hydrogen or (C₁-C₈)alkyl;
R³ represents hydrogen or (C₁-C₈)alkyl, provided that R² and R³ are not hydrogen at the same time;
R⁴ represents hydrogen, halogen, (C₁-C₄)alkyl or (C₁-C₄)haloalkyl;
comprising the steps (a), (b) and (c), wherein in
step (a) a compound of the formula (II)

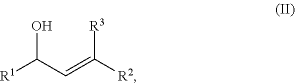

in which the definitions of the substituents R¹, R² and R³ listed in the formula (II) are the same as in the formula (I), is converted in the presence of a activating agent to a compound of the formula (III)

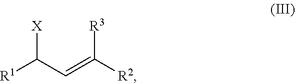

in which X represents a halogen or O—SO₂R wherein R is a methyl, phenyl or tolyl group and in which the definitions of the substituents R¹, R² and R³ listed in the formula (III) are the same as in the formula (I), and wherein in
step (b) the compound of the formula (III) is reacted with a compound of the formula (IIIa)

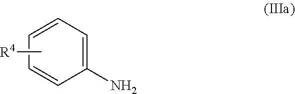

in which the definition of the substituent R⁴ listed in the formula (IIIa) is the same as in the formula (I) to obtain a compound of the formula (IV)

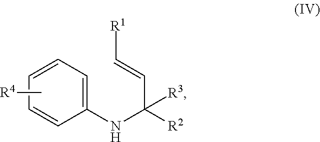

in which the definitions of the substituents R¹, R², R³ and R⁴ listed in the formula (IV) are the same as in the formula (I), and comprising
(c) the rearrangement of the compound of the formula (IV) in the presence of an acid to obtain the compound of the formula (I).

Furthermore the present invention provides a process for the preparation of a compound of the formula (V)

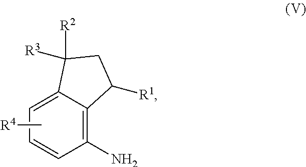

in which
R¹ represents (C₁-C₄)alkyl;
R² represents hydrogen or (C₁-C₈)alkyl;
R³ represents hydrogen or (C₁-C₈)alkyl, provided that R² and R³ are not hydrogen at the same time;
R⁴ represents hydrogen, halogen, (C₁-C₄)alkyl or (C₁-C₄)haloalkyl,
comprising the steps (b), (c) and (d),
wherein in a step (b) a compound of the formula (III)

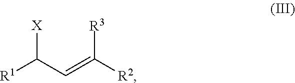

in which X represents a halogen or O—SO₂R wherein R is a methyl, phenyl or tolyl group and wherein the definitions of the substituents R¹, R² and R³ listed in the formula (III) are the same as in the formula (V)
is reacted with a compound of the formula (IIIa)

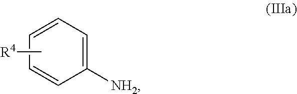

in which the definition of the substituent R⁴ listed in the formula (IIIa) is the same as in the formula (V), to obtain a compound of the formula (IV)

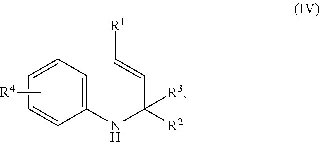

in which the definitions of the substituents R¹, R², R³ and R⁴ listed in the formula (IV) are the same as in the formula (V),
and wherein in a step (c) the compound of the formula (IV), wherein the definitions of the substituents R¹, R², R³ and R⁴ listed in the formula (IV) are the same as in the formula (V), is rearranged in the presence of an acid to obtain the compound of the formula (I)

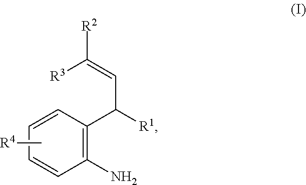

wherein the definitions of the substituents R¹, R², R³ and R⁴ listed in the formula (I) are the same as in the formula (V),
and wherein in a step (d) the compound of the formula (I) is reacted with aqueous sulfuric acid or anhydrous hydrogen fluoride (HF), to yield a compound of formula (V).

Furthermore the present invention provides a process for the preparation of a compound of the formula (V)

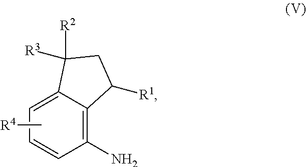

in which
R¹ represents (C₁-C₄)alkyl;
R² represents hydrogen or (C₁-C₈)alkyl;
R³ represents hydrogen or (C₁-C₈)alkyl, provided that R² and R³ are not hydrogen at the same time;
R⁴ represents hydrogen, halogen, (C₁-C₄)alkyl or (C₁-C₄)haloalkyl, comprising the steps (a), (b), (c) and (d), wherein in step (a) a compound of the formula (II)

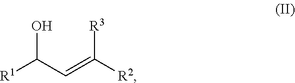

in which the definitions of the substituents R¹, R² and R³ listed in the formula (II) are the same as in the formula (I), is converted in the presence of a activating agent to a compound of the formula (III)

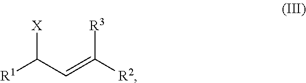

in which X represents a halogen or O—SO₂R wherein R is a methyl, phenyl or tolyl group and in which the definitions of the substituents R¹, R² and R³ listed in the formula (III) are the same as in the formula (I), and wherein in
step (b) the compound of the formula (III) is reacted with a compound of the formula (IIIa)

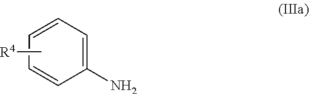

in which the definition of the substituent R⁴ listed in the formula (IIIa) is the same as in the formula (I) to obtain a compound of the formula (IV)

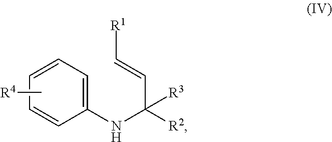

in which the definitions of the substituents R¹, R², R³ and R⁴ listed in the formula (IV) are the same as in the formula (I), and comprising (c) the rearrangement of the compound of the formula (IV) in the presence of an acid to obtain the compound of the formula (I)

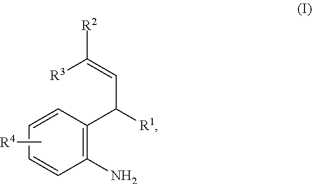

wherein the definitions of the substituents R¹, R², R³ and R⁴ listed in the formula (I) are the same as in the formula (V),
and wherein in a step (d) the compound of the formula (I) is reacted with aqueous sulfuric acid or anhydrous hydrogen fluoride (HF), to yield a compound of formula (V).

Preferred, particularly preferred and most preferred definitions of the residues R¹, R², R³ and R⁴ listed in the herein-defined formulae (I), (II), (III), (IIIa), (IV) and (V) (further referred to (I)-(V)) are elucidated below.

It is preferable when in each case:

X represents halogen or O—SO₂R wherein R is a methyl, phenyl or tolyl group;
R¹ represents (C₁-C₄)alkyl;
R² represents hydrogen or (C₁-C₈)alkyl;
R³ represents hydrogen or (C₁-C₈)alkyl, provided that R² and R³ are not hydrogen at the same time;
R⁴ represents hydrogen, halogen, (C₁-C₄)alkyl or (C₁-C₄)haloalkyl;
provided that if R¹ and R² are both the same, then R³ is not hydrogen and provided that if R¹ and R³ are both the same, then R² is not hydrogen.

It is also preferable when in each case:

X represents halogen;
R¹ represents methyl or n-propyl;
R² and R³ represent methyl;
R⁴ represents hydrogen or fluorine.

It is also preferable when in each case:

R¹ represents methyl or n-propyl;
R² and R³ represent methyl;
R⁴ represents hydrogen or fluorine.

It is particularly preferable when in each case:

X represents halogen;
R¹ represents methyl or n-propyl;
R² and R³ represent methyl;
R⁴ represents hydrogen.

It is also particularly preferable when in each case:

R¹ represents methyl or n-propyl;
R² and R³ represent methyl;
R⁴ represents hydrogen.

It is most preferable when in each case:

X represents bromine;
R¹ represents n-propyl;
R² and R³ represent methyl;
R⁴ represents hydrogen.

It is also most preferable when in each case:

R¹ represents n-propyl;
R² and R³ represent methyl;
R⁴ represents hydrogen.

It is also most preferable when in each case:

X represents bromine;
R¹, R² and R³ represent methyl;
R⁴ represents hydrogen.

It is also most preferable when in each case:

R¹, R² and R³ represent methyl;
R⁴ represents hydrogen.

It is also most preferable when in each case:

X represents bromine;
R¹, R² and R³ represent methyl;
R⁴ represents fluorine.

It is also most preferable when in each case:

R¹, R² and R³ represent methyl;
R⁴ represents fluorine.

Unless otherwise stated, the following definitions apply for the substituents and residues used throughout this specification and claims:

Halogen: fluorine, chlorine, bromine or iodine, preferably fluorine, chlorine or bromine, particularly preferably fluorine or chlorine, more preferably chlorine or bromine and most preferably bromine.

Alkyl: saturated, straight-chain or branched hydrocarbyl radical having 1 to 8, preferably 1 to 6, and more preferably 1 to 4 carbon atoms, for example (but not limited to) C₁-C₆-alkyl such as methyl, ethyl, propyl (n-propyl), 1-methylethyl (iso-propyl), butyl (n-butyl), 1-methylpropyl (sec-butyl), 2-methylpropyl (iso-butyl), 1,1-dimethylethyl (tert-butyl), pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Particularly, said group is a C1-C4-alkyl group, e.g. a methyl, ethyl, propyl, 1-methylethyl (isopropyl), butyl, 1-methylpropyl (sec-butyl), 2-methylpropyl (iso-butyl) or 1,1-dimethylethyl (tert-butyl) group.

Haloalkyl: straight-chain or branched alkyl groups having 1 to 8, preferably 1 to 6 and more preferably 1 to 4 carbon atoms (as specified above), where some or all of the hydrogen atoms in these groups are replaced by halogen atoms as specified above, for example (but not limited to) C₁-C₃-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl. Tolyl: o-tolyl, m-tolyl or p-tolyl

The term telescoping synthesis is defined as a sequence of different chemical transformations which ultimately leads to the isolation of a product and runs through several different intermediates whose isolation is omitted in order to maximize the space-time-yield of the process. Minimal downstreaming operations such as liquid-liquid extraction and distillation may be implemented between the chemical transformations in order to remove substances not being compatible with the follow-up chemistry.

### DETAILED DESCRIPTION OF THE PROCESS

The process according to the invention can be conducted as shown in schemes (1) to (3):

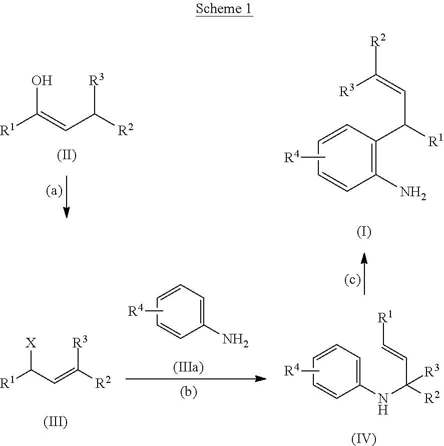

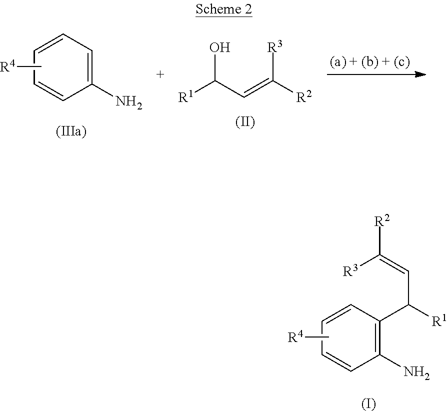

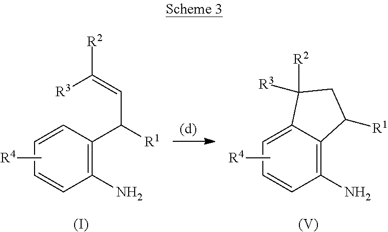

In scheme 1 the substituents X, R¹, R², R³ and R⁴ of the formulae (I)-(IV) each have the general, preferred, particularly preferred, more preferred or most preferred meanings which have already been defined for these substituents in connection with the description of the compounds of the formulae (I) to (IV).

In scheme 2 the substituents R¹, R², R³ and R⁴ of the formulae (II), (IIIa) and (I) each have the general, preferred, particularly preferred, more preferred or most preferred meanings which have already been defined for these substituents in connection with the description of the compounds of the formulae (I) to (IV).

In scheme 3 the substituents R¹, R², R³ and R⁴ of the formulae (I) and (V) each have the general, preferred, particularly preferred, more preferred or most preferred meanings which have already been defined for these substituents in connection with the description of the compounds of the formulae (I) and (V).

The process shown in scheme 1 is the process according to the invention when conducted stepwise, i.e. the steps (a), (b) and (c) are performed consecutively.

The process shown in scheme 2 is the process according to the invention when conducted as a telescoping synthesis. This means, the process is worked as a sequential one-pot synthesis with reagents added to a reactor one at a time and wherein minimal work-up procedures are performed during the process.

The reagents used and reaction conditions applied in the processes shown in scheme 1 and 2 are both the same.

To obtain the compound of the formula (II), an alkyl- or alkenylaldehyde is dosed neat or diluted to a stirred Grignard solution containing an C₁-C₄-alkenyl- or C₁-C₄-alkylmagnesium halide. The principle of this reaction is known from the literature, e. g. Adam's protocol and is applicable also in this case (W. Adam, V. R. Stegmann, Synthesis 2001, p 1203-1214).

### Step (a)

In step (a) an allylalcohol of the formula (II) is activated via transformation of the hydroxyl group into a suitable leaving group X, generating a compound of the formula (III). This is achieved by addition of an activating reagent to the diluted or undiluted allylalcohol at a suitable temperature.

Preferably, the activating reagent is added to the diluted or undiluted allylalcohol in stoichiometric amounts.

Preferably, the activating agent in step (a) is selected from anhydrous hydrogen chloride, anhydrous hydrogen bromide, thionyl chloride, phosphoroxychloride, phosphorus trichloride, phosphorus tribromide (PBr₃), methanesulphonic chloride, methanesulphonic anhydride, 4-toluenesulphonic chloride and 4-toluenesulphonic anhydride.

Particularly preferably, the activating agent is selected from anhydrous hydrogen chloride, anhydrous hydrogen bromide, thionyl chloride, phosphoroxychloride, phosphorus trichloride, and phosphorus tribromide.

More preferably, the activating agent is phosphorus tribromide.

Generally, step (a) can be conducted without the presence of a solvent or in one or more of the following solvents: ethers such as tetrahydrofuran (THF), dioxane, diethyl ether, diglyme, methyl tert-butyl ether (MTBE), tert-amyl methyl ether (TAME), dimethyl ether, 2-methyl-THF; alkanes or cycloalkanes or alkyl-substituted cycloalkanes, for example n-hexane, n-heptane, cyclohexane, isooctane or methylcyclohexane; nitriles such as acetonitrile (ACN) or butyronitrile; aromatic hydrocarbons such as toluene, xylenes, anisole, mesitylene; esters such as ethyl acetate, isopropyl acetate, butyl acetate, pentyl acetate; halogenated aromatic hydrocarbons, particularly chlorohydrocarbons such as tetrachloroethylene, tetrachloroethane, dichloropropane, methylene chloride (dichloromethane, DCM), dichlorobutane, chloroform, trichloroethane, trichloroethylene, pentachloroethane, difluorobenzene, trifluorobenzene, 1,2-dichloroethane, chlorobenzene, bromobenzene, dichlorobenzene, especially 1,2-dichlorobenzene, chlorotoluene, trichlorobenzene; fluorinated aliphatic and aromatic compounds such as trichlorotrifluoroethane, benzotrifluoride and 4-chlorobenzotrifluoride. It is also possible to use solvent mixtures.

Preferably, the solvent is an aromatic solvent.

Also preferably, the solvent is selected from tetrahydrofuran, n-heptane, toluene, xylenes, anisole, trifluorobenzene and chlorobenzene.

Particularly preferably, the solvent is selected from chlorobenzene, toluene, xylene, anisole and trifluorobenzene.

More preferably, the solvent is selected from xylenes, anisole, and chlorobenzene.

Most preferably, the solvent is chlorobenzene.

Preferably, step (a) of the process according to the invention is carried out at a temperature in the range of from −5° C. to 120° C.

Particularly preferably, step (a) of the process according to the invention is carried out at a temperature in the range of from 0° C. to 60° C.

More preferably, step (a) of the process according to the invention is carried out at a temperature in the range of from 0° C. to 40° C.

Most preferably, step (a) of the process according to the invention is carried out at a temperature in the range of from 0° C. to 20° C.

### Step (b)

To obtain the compound of the formula (IV) via step (b), the compound of the formula (III) is generally reacted with the compound of the formula (IIIa) in the presence of a base and a solvent at a suitable temperature.

Preferably, the compound of the formula (III) is reacted with the compound of the formula (IIIa) in stoichiometric amounts.

Suitable bases are all customary inorganic or organic bases. These preferably include alkaline earth metal or alkali metal hydrides, hydroxides, amides, alkoholates, acetates, carbonates or bicarbonates, such as, for example, sodium acetate, sodium carbonate, potassium carbonate, potassium bicarbonate, sodium bicarbonate or ammonium carbonate, and also tertiary amines, such as trimethylamine, triethylamine, tri-n-butylamine, N,N-dimethylaniline, N,N-dimethylbenzylamine, pyridine, N-methylpiperidine, N-methylmorpholine, N,N-dimethylaminopyridine, 1,4-Diazabicyclo[2.2.2]octane (DABCO), 1,5-Diazabicyclo[4.3.0]non-5-ene (DBN) or 1,8-diazabicyclo[5.4.0]-undec-7-ene (DBU), N,N-diisopropylethylamine, N,N,N′,N′-tetramethylguanidine, N-Methylimidazole.

Particularly preferably, the base in step (b) is selected from N-methylmorpholine, N,N-diisopropylethylamine, N,N,N′,N′-tetramethylguanidine, tri-n-butylamine, triethylamine, DABCO, DBU and N-Methylimidazole.

More preferably, the base used in step (b) is selected from N-methylmorpholine, N,N-diisopropylethylamine, N,N,N′,N′-tetramethylguanidine, tri-n-butylamine and triethylamine.

Most preferably, the base used in step (b) is N-methylmorpholine or N,N-diisopropylethylamine.

Step (b) is preferably conducted in one or more of the solvents listed in the general solvent definition of step (a).

Particularly preferably, the solvent is selected from tetrahydrofuran, n-heptane, chlorobenzene, toluene, xylenes, anisole and trifluorobenzene.

More preferably, the solvent is selected from chlorobenzene, toluene, xylene, anisole and trifluorobenzene.

Even more preferably, the solvent is selected from chlorobenzene, xylenes and anisole.

Most preferably, the solvent is chlorobenzene.

Preferably, step (b) of the process according to the invention is carried out at a temperature in the range of from 10° C. to 90° C.

Particularly preferably, step (b) of the process according to the invention is carried out at a temperature in the range of from 15° C. to 50° C.

More preferably, step (b) of the process according to the invention is carried out at a temperature in the range of from 20° C. to 30° C.

Step (c) To obtain the compound of the formula (I) via step (c), the compound of the formula (IV) is generally reacted in the presence of a Lewis or Brønsted acid and a solvent at a suitable temperature.

Preferably, the compound of the formula (IV) is reacted in the presence of catalytic to stoichiometric amounts of the Lewis or Brønsted acid.

Generally, step (c) according to the invention is carried out in the presence of a suitable Lewis acid, for example metal halides like AlCl₃, BF₃ and other Lewis acids known in literature; or triflates, for example silver triflate, zinc trifluoromethanesulfonate (Zn(OTf)₂), Copper(II)trifluoromethanesulfonate (Cu(OTf)₂), nickel(II)trifluoromethanesulfonate (Ni(OTf)₂), Iron(II) trifluoromethanesulfonate (Fe(OTf)₂,), Iron(III) trifluoromethanesulfonate (Fe(OTf)₃) and other triflates described in the literature. The process may also be carried out in the presence of Bronstedt acids like e.g. HCl, HBr, HF, H₂SO₄, KHSO₄, AcOH, H₃NSO₃, trifluoroacetic acid, p-toluenesulfonic acid, methanesulfonic acid, camphorsulfonic acid, methansulfonic acid, benzenesulfonic acid, trifluoromethansulfonic acid, polyphosphoric acid, phosphoric acid, phenylphosphonic acid, ethylphosphonic acid, acetic acid, chloroacetic acid, dichloroacetic acid, trichloroacetic acid and trifluoroacetic acid.

Preferably, in the process according to the invention the acid in step (c) is selected from 4-toluenesulfonic acid, methanesulfonic acid, trifluoromethanesulfonic acid, zinc trifluoromethanesulfonate (Zn(OTf)₂), Copper(II)trifluoromethanesulfonate (Cu(OTf)₂), nickel(II)trifluoromethanesulfonate (Ni(OTf)₂), Iron(II) trifluoromethanesulfonate (Fe(OTf)₂,), Iron(III) trifluoromethanesulfonate (Fe(OTf)₃), benzenesulfonic acid, H₂SO₄, H₃NSO₃, phenylphosphonic acid, ethylphosphonic acid, phosphoric acid, acetic acid, chloroacetic acid, dichloroacetic acid, trichloroacetic acid and trifluoroacetic acid.

More preferably the acid in step (c) is methanesulfonic acid or 4-toluenesulphonic acid.

Most preferably the acid in step (c) is 4-toluenesulphonic acid.

Step (c) is conducted in one or more of the solvents listed in the general solvent definition of step (a).

Preferably, the solvent is selected from chlorobenzene, toluene, xylenes, anisole and trifluorobenzene.

Particularly preferably, the solvent is selected from chlorobenzene, xylenes and anisole.

More preferably, the solvent is chlorobenzene.

Preferably, step (c) of the process according to the invention is carried out at a temperature in the range of from 80° C. to 140° C.

During the condensation of the substituted aniline of the formula (IIIa) and the activated allylalcohol of the formula (III), desired and undesired regioisomers of the N-allylated intermediates are generated, wherein the compound of the formula (IV) is the desired N-allylated intermediate. During the subsequent ACR only the N-allylated intermediate of the formula (IV) rearranges to the compound of the formula (I) but not the undesired regioisomer. After the ACR, the undesired regioisomers can be eliminated by a temperature-controlled degradation, preferably into the separable organic compounds aniline and diene.

Therefore particularly preferably, step (c) is carried out consecutively first at a temperature in the range of from 80° C. to 95° C. and second at a temperature in the range of from 115° C. to 140° C. The first temperature range is ideal for the ACR to generate the compound of the formula (I). The second temperature range is ideal for the degradation of the undesired regioisomer into organic compounds such as aniline and diene. Those are easily separable from the final product (i.e. the compound of the formula (I)) as aniline can easily be washed off the product phase with acidic water. The diene can also be simply removed via distillation due to its much lower boiling point compared to the final product.

More preferably step (c) is carried out consecutively first at a temperature in the range of 85 to 90° C. and second at a temperature in the range of 125° C. to 130° C.

In a preferred embodiment of the invention, the steps (a), (b) and (c) of the process according to the invention are conducted consecutively in a telescoping synthesis as defined above by utilizing the same solvent for all of the steps (a), (b) and (c). Particularly preferably, the solvent used in the telescoping synthesis is chlorobenzene.

In a further preferred embodiment, during the process according to the invention the product of step (a) is not isolated before conducting step (b) and/or the product of step (b) is not isolated before conducting step (c).

The present invention further relates to a process for producing a compound of the formula (V)

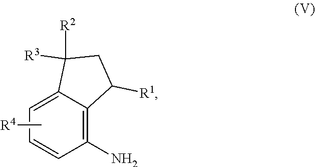

wherein in formula (V) the substituents R¹, R², R³ and R⁴ each have the general, preferred, particularly preferred, more preferred or most preferred meanings which have already been defined for these substituents in connection with the description of the compounds of the formulae (I)-(IV) as defined above, comprising the steps (b) and (c) or comprising the steps (a), (b) and (c) as defined above and further comprising step (d), wherein the compound of the formula (I) is cyclized in the presence of an acid to obtain the compound of the formula (V).

Suitable acids for step (d) are sulphonic acids, in particular trifluoromethanesulphonic acid (TfOH), methanesulphonic acid (MsOH) and polyphosphoric acid as known from WO 2017/133981.

### Step (d)

To obtain the compound of the formula (V) according to the invention and as shown in scheme 3, the compound of the formula (I) is reacted with aqueous sulfuric acid or anhydrous hydrogen fluoride (HF), wherein the definitions of the substituents R¹, R², R³ and R⁴ of the formulae (V) and (I) each have the general, preferred, particularly preferred, more preferred or most preferred meanings which have already been defined for these substituents in connection with the description of the compounds of the formulae (V) and (I).

Preferably, when aqueous sulfuric acid is utilized as the mediator, the compound of the formula (I) and the aqueous sulfuric acid are dosed simultaneously into an empty reaction vessel. If said reaction vessel requires a minimum filling level, it can be filled with aqueous sulfuric acid up to this level. Simultaneous dosing of the substrate (i.e. the compound of the formula (I)) and aqueous sulfuric acid maintains high chemoselectivity throughout the entire reaction due to keeping the substrate concentration at a constant level. Advantageously, this prevents oligo- and polymerization of the substrate. When anhydrous hydrogen fluoride is utilized as the mediator this tendency towards oligo- and polymerization is not observed. Preferably the substrate is dosed to anhydrous hydrogen fluoride in this case.

Preferably, the process according to the invention is carried out at a temperature in the range of from −80° C. to 30° C., particularly preferably at a temperature in the range of from −50° C. to 20° C., more preferably at a temperature in the range of from −30° C. to 20° C.

Also preferably, if aqueous sulfuric acid is used as cyclization mediator, the process according to the invention is carried out at a temperature in the range of from 0° C. to 25° C., particularly preferably, at a temperature in the range of from 0° C. to 20° C., more preferably, at a temperature in the range of from 0° C. to 15° C.

Also preferably, if anhydrous hydrogen fluoride is used as cyclization mediator, the process according to the invention is carried out at a temperature in the range of from −80° C. to 20° C., particularly preferably at a temperature in the range of from −50° C. to 20° C., more preferably at a temperature in the range of from −30° C. to 20° C.

The process is generally conducted at normal pressure or at elevated pressure in an autoclave.

Preferably, the aqueous sulfuric acid used in the process according to the invention has a concentration of at least 85 w %. Particularly preferably, the aqueous sulfuric acid used in the process according to the invention has a concentration in the range of from 85 w % to 95 w %, more preferably in the range of from 88 w % to 92 w %, most preferably the concentration of the aqueous sulfuric acid is 90 w %.

The amount of the employed cyclization mediator may be varied over a wide range but is preferably in the range of from 3-45 molar equivalents, preferably of from 6 to 40 molar equivalents, especially preferably of from 9 to 35 molar equivalents based on the total amount of the compound of the formula (I).

If aqueous sulfuric acid is used as cyclization mediator, its used amount may be varied over a wide range but is preferably in the range of from 3-18 molar equivalents, preferably of from 6 to 15 molar equivalents, especially preferably of from 9 to 12 molar equivalents based on the total amount of the compound of the formula (II).

If anhydrous hydrogen fluoride is used as cyclization mediator, its used amount may be varied over a wide range but is preferably in the range of from 15-45 molar equivalents, preferably of from 20-40 molar equivalents, especially preferably of from 25-35 molar equivalents based on the total amount of the compound of the formula (I).

Generally, the process according to the invention can be conducted in the absence of a solvent or in the presence of one or more of the following solvents: alkanes or cycloalkanes or alkyl-substituted cycloalkanes, for example n-hexane, n-heptane, cyclohexane, isooctane or methylcyclohexane; aromatic hydrocarbons such as toluene, xylenes, mesitylene; amides such as N,N-dimethylacetamide (DMAc), N,N-dimethylformamide (DMF), N-methylpyrrolidone; halohydrocarbons and halogenated aromatic hydrocarbons, particularly chlorohydrocarbons such as tetrachloroethylene, tetrachloroethane, dichloropropane, methylene chloride (dichloromethane, DCM), dichlorobutane, chloroform, trichloroethane, pentachloroethane, difluorobenzene, trifluorobenzene, 1,2-dichloroethane, chlorobenzene, bromobenzene, dichlorobenzene, especially 1,2-dichlorobenzene, chlorotoluene, trichlorobenzene; fluorinated aliphatic and aromatic compounds such as trichlorotrifluoroethane, benzotrifluoride, 4-chlorobenzotrifluoride. It is also possible to use solvent mixtures.

Preferably, the process is carried out in the absence of a solvent, using aqueous sulfuric acid or anhydrous HF as a mediator of the cyclization reaction and as solvent.

Preferably, when HF is used as the cyclization mediator in the process according to the invention, HF is used in anhydrous form, optionally as solution in organic solvents, more preferably HF is used in anhydrous form with a boiling point of 20° C. (i.e. without any organic solvents and free of water).

The reaction time in aqueous sulfuric acid or anhydrous HF is not critical and it can generally be varied from 1 to 30 hours (h), preferably from 3 to 24 h.

According to the invention the starting material, i.e. the compound of the formula (I), is mixed with aqueous sulfuric acid or anhydrous HF and stirred for a certain time at a certain temperature as defined above. For the isolation of the product, the excess of sulfuric acid is removed via addition of water leading to precipitation of the ammonium hydrogensulfate salt of (I), subsequent filtration and washing the salt with water. In order to recycle the sulfuric acid, the filtrate can be subjected to a distillation to obtain the required acid concentration. Advantageously, anhydrous hydrogen fluoride can be recycled more easily as it can be distilled from the reaction solution directly, leaving the ammonium fluoride salt of (V). Said salts are neutralized with a suitable base and extracted into a suitable organic solvent, from which compound (V) is isolated via removal of the solvent by distillation followed by purification via high vacuum distillation.

The present invention further relates to a process for producing a compound of the formula (VII)

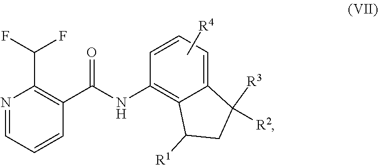

wherein in formula (VII) the substituents R¹, R², R³ and R⁴ each have the general, preferred, particularly preferred, more preferred or most preferred meanings which have already been defined for these substituents in connection with the description of the compounds of the formulae (I)-(IV) as defined above, comprising the steps (b) to (d) or comprising the steps (a) to (d) as defined above and further comprising step (e), wherein the compound of the formula (V) is reacted with a compound of the formula (VI)

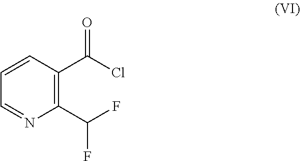 [SMILES: O=C(Cl)c1cccnc1C(F)F]

to obtain the compound of the formula (VII). The reaction according to step (e) is in principle known from e.g. WO 2014/095675 A1.

Depending on the type of substituents, the compounds according to the invention can occur as geometric and/or optical isomers or as their corresponding isomeric mixtures in various compositions. These isomers are, for example, enantiomers, diastereomers or geometric isomers. As a consequence, the invention described herein includes both the pure stereoisomers and every mixture of these isomers.

Another object of the present invention is the compound of the formula (III)

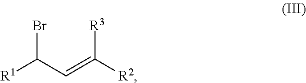

wherein in formula (III) the substituents R¹, R² and R³ each have the general, preferred, particularly preferred, more preferred or most preferred meanings which have already been defined for these substituents in connection with the description of the compounds of the formulae (I)-(IV) as defined above.

Another object of the present invention is the compound of the formula (IV)

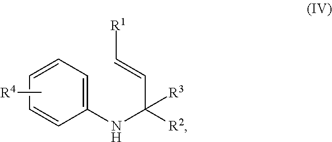

wherein in formula (IV) the substituents R¹, R², R³ and R⁴ each have the general, preferred, particularly preferred, more preferred or most preferred meanings which have already been defined for these substituents in connection with the description of the compounds of the formulae (I)-(IV) as defined above.

The present invention is elucidated in detail by the examples which follow, although the examples should not be interpreted in such a manner that they restrict the invention.

### PREPARATION EXAMPLES

### Example 1

### Preparation of rac-4-bromo-2-methyl-hept-2-ene (a compound according to formula (III))

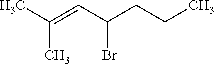

In a 25 mL three-necked reaction flask equipped with a thermometer was placed 2 mL of anhydrous THF under a gentle stream of argon. 5.0 g (90% purity, 35.1 mmol, 1.0 eq) of rac-2-methylhept-2-en-4-ol were added in one portion. The solution was cooled to 0° C. Then 1.1 mL (3.17 g, 11.6 mmol, 0.33 eq) of phosphorus tribromide were dosed to the solution at 0° C. for 10 minutes via syringe pump. Afterwards the reaction mixture was allowed to reach 22° C. To the mixture was added another 2 mL of anhydrous THF. The resulting two liquid phases were separated and the lower one was discarded. The upper phase was liberated from volatile components via distillation at 40° C. down to a vacuum of 20 mbar to leave 6.27 g (92% purity, 30.2 mmol, 86% yield) of rac-4-bromo-2-methyl-hept-2-ene as a dark yellow liquid. ¹H-NMR (400 MHz; CDCl₃) δ=5.43-5.39 (m, 1H), 4.88-4.82 (m, 1H), 1.88-1.77 (m, 2H, H5), 1.75 (s, 3H), 1.71 (s, 3H), 1.45-1.35 (m, 2H), 0.93 (t, J=7.4 Hz, 3H).

### Example 2

### Preparation of N-[(E)-1,1-dimethylhex-2-enyl]aniline (a compound according to formula (IV))

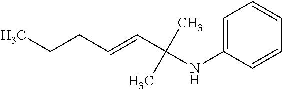

In a 500 mL four-necked reaction flask equipped with a thermometer was placed 150.0 g (88% purity, 1.03 mol, 1.0 eq) of rac-2-methylhept-2-en-4-ol under a gentle stream of argon. Then 33.5 mL (95.6 g, 0.35 mol, 0.34 eq) of phosphorus tribromide were dosed to the solution at 5° C. over a period of 3 hours via syringe pump. Afterwards the reaction mixture was allowed to reach 22° C. To the mixture was added 60 mL of anhydrous THF. The resulting two liquid phases were separated and the lower one was discarded. The upper phase was dosed to a solution comprising 94.7 mL (96.7 g, 1.03 mol, 1.0 eq) of aniline, 125.6 mL (115.6 g, 1.13 mol, 1.1 eq) of 4-methylmorpholine in 920 mL anhydrous THF at 22° C. over a period of 1.5 hours. A yellow solution with white precipitate was obtained. After completed dosing the mixture was diluted with 100 mL of deionized water. Afterwards the volatiles of this mixture were removed via distillation at 40° C. down to a vacuum of 50 mbar. The distillation residue was diluted with 900 mL of MTBE and 1200 mL of deionized water. Phases were separated and the organic phase was dried with 1000 mL of saturated brine and over magnesium sulphate. The drying agent was filtered off and the filtrate was concentrated at 40° C. down to a vacuum of 4 mbar to leave 193.1 g (87.5% purity, 0.83 mol, 81% yield) of a red oil containing N-[(E)-1,1-dimethylhex-2-enyl]aniline and rac-N-(3-methyl-1-propyl-but-2-enyl)aniline in a regioisomeric ratio of 84:16. Spectral data of the major regioisomer N-[(E)-1,1-dimethylhex-2-enyl]aniline: ¹H-NMR (400 MHz; CDCl₃) δ=7.11-7.07 (m, 2H), 6.71-6.69 (m, 3H), 5.58-5.57 (m, 2H), 3.63 (bs, 1H), 2.03-2.01 (m, 2H), 1.40 (q, J=8.0 Hz, 2H), 1.37 (s, 6H), 0.89 (t, J=8.0 Hz, 3H). The regioisomeric ratio of 84:16 was determined independently via HPLC analysis and via comparison of the integration values of the major isomer's singulett of the geminal methyl groups (singulett at 1.37 ppm) with the two singletts of the geminal methyl groups of the minor isomer rac-N-(3-methyl-1-propyl-but-2-enyl)aniline (singuletts at 1.74 ppm and 1.70 ppm) in ¹H-NMR.

### Example 3

### Preparation of rac-2-(3-methyl-1-propyl-but-2-enyl)aniline (a compound according to formula (I))

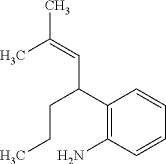

To a 100 mL four-necked round-bottomed flask equipped with a thermometer was added 36.1 mL of chlorobenzene and 10.0 g (88.5% purity, 43.53 mmol, 1.0 eq) of a regioisomeric mixture of N-[(E)-1,1-dimethylhex-2-enyl]aniline and rac-N-(3-methyl-1-propyl-but-2-enyl)aniline with an isomeric ratio of 84:16. The resulting solution was degassed via argon-bubbling for 15 minutes at 22° C. Afterwards 2.1 g (10.88 mmol, 0.25 eq) of 4-toluenesulphonic acid monohydrate was added in one portion. The mixture was heated to 90° C. internal temperature to form a solution, which was stirred for 5 hours at this temperature until a HPLC measurement indicated complete conversion of N-[(E)-1,1-dimethylhex-2-enyl]aniline. Afterwards the temperature was increased until reflux at 130° C. internal temperature was obtained. After further stirring for 1.5 hours a HPLC measurement indicated complete degradation of rac-N-(3-methyl-1-propyl-but-2-enyl)aniline. The reaction mixture was cooled down to 22° C. A white precipitate was formed. To the reaction mixture was then added 50 mL of chlorobenzene and 100 mL of deionized water. The phases were separated and the organic phase was washed with 20 mL of deionized water. Afterwards the organic phase was concentrated at 60° C. and down to a vacuum of 5 mbar to leave 7.1 g (74% purity, 25.80 mmol, 59% yield) of a clear orange oil. ¹H-NMR (400 MHz; CDCl₃) δ=7.12 (d, J=7.7 Hz, 1H), 7.01 (dd, J=7.5, 7.7 Hz, 1H), 6.77 (dd, J=7.5, 7.7 Hz, 1H), 6.65 (d, J=7.7 Hz, 1H), 5.12-5.08 (m, 1H), 3.56 (bs, 2H), 3.47-3.41 (m, 1H), 1.71 (s, 6H), 1.65-1.56 (m, 2H), 1.40-1.26 (m, 2H), 0.93 (t, J=8.0 Hz, 3H).

### Example 4

### Preparation of rac-2-(3-methyl-1-propyl-but-2-enyl)aniline (a Compound According to Formula (I)) Via a Telescoped Reaction

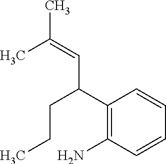

In a 1000 mL four-necked reaction flask equipped with a thermometer was dissolved 250.0 g (89% purity, 1.73 mol, 1.0 eq) of rac-2-methylhept-2-en-4-ol in 250.0 g of chlorobenzene under a gentle stream of argon. Then 54.8 mL (156.06 g, 0.57 mol, 0.33 eq) of phosphorus tribromide were dosed to the solution at 3° C. over a period of 4 hours via syringe pump. Afterwards the reaction mixture was allowed to reach 22° C. Phases were separated and the lower, dark phase was discarded. The yellow-greenish upper phase was then dosed to a 4000 mL jacketed reactor containing a stirred solution of 151.2 mL (154.6 g, 1.64 mol, 0.95 eq) of aniline and 182.5 mL (167.9 g, 1.64 mol, 0.95 eq) of 4-methylmorpholine in 1031 mL chlorobenzene at 24° C. under argon over a period of 2 hours. The resulting suspension was then stirred for 1 hour at 24° C. To the reaction mixture were then added 1800 mL of saturated brine and 600 ml of deionized water. The resulting two liquid phases were mixed and separated. The lower phase was drained off and discarded. Afterwards the organic phase was degassed via argon bubbling for 0.5 hours. To the reaction solution was then added 82.3 g (0.43 mol, 0.25 eq) of 4-toluenesulphonic acid monohydrate in one portion. The mixture was heated to 90° C. internal temperature to form a solution, which was stirred for 5 hours at this temperature until a HPLC measurement indicated complete conversion of N-[(E)-1,1-dimethylhex-2-enyl]aniline. Afterwards the temperature was increased until reflux at 130° C. internal temperature was obtained. After further stirring for 1.5 hours a HPLC measurement indicated complete degradation of rac-N-(3-methyl-1-propyl-but-2-enyl)aniline. The reaction mixture was cooled down to 22° C. A white precipitate was formed. To the reaction mixture was then added 1000 mL of deionized water. The phases were mixed and separated The lower phase was drained off and discarded. Afterwards the organic phase was concentrated at 60° C. and down to a vacuum of 5 mbar to leave 258.0 g (76% purity, 0.96 mol, 55% yield) of a dark red oil. ¹H-NMR (400 MHz; CDCl₃) δ=7.12 (d, J=7.7 Hz, 1H), 7.01 (dd, J=7.5, 7.7 Hz, 1H), 6.77 (dd, J=7.5, 7.7 Hz, 1H), 6.65 (d, J=7.7 Hz, 1H), 5.12-5.08 (m, 1H), 3.56 (bs, 2H), 3.47-3.41 (m, 1H), 1.71 (s, 6H), 1.65-1.56 (m, 2H), 1.40-1.26 (m, 2H), 0.93 (t, J=8.0 Hz, 3H).

### Example 5

### Preparation of rac-2-(1,3-dimethyl-but-2-enyl)aniline (a Compound According to Formula (I)) Via a Telescoped Reaction

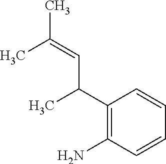

In a 100 mL four-necked reaction flask equipped with a thermometer was placed 20.00 g (98.5% purity, 196.69 mmol, 1.00 eq) of rac-4-methylpent-3-en-2-ol and 50 mL of chlorobenzene. The solution was cooled to 0° C. and inertized via Argon. To the solution was dosed 17.75 g (99% purity, 64.91 mmol, 0.33 eq) of phosphorus tribromide at 0-4° C. over a period of 90 minutes. After 15 minutes of post-stirring at 0° C. the reaction mixture was allowed to warm up to 22° C. and was subsequently transferred into a dropping funnel. Of the two separated phases the dark viscous lower layer was discarded. A second 100 mL four-necked reaction flask was equipped with a thermometer and with the dropping funnel containing the product phase of the first reaction. Then 17.58 g (99% purity, 186.85 mmol, 0.95 eq) of aniline and 19.09 g (99% purity, 186.85 mmol, 1.00 eq) of N-methylmorpholine were dissolved in 50 mL of chlorobenzene at 22° C. To this solution was dosed the product solution of the first reaction within 2 hours maintaining an internal temperature of 22-24° C. via waterbath cooling. After 1 hour of post-stirring at 22° C. the organic phase was washed with 2×200 mL deionized water and was subsequently degassed with Argon for 1 hour. To the organic phase was then added 1.23 g (99% purity, 6.39 mmol, 3.3 mol %) of 4-toluenesulfonic acid monohydrate in one portion at 22° C. Afterwards the reaction mixture was heated to 90° C. internal temperature and stirred for 6 hours until HPLC monitoring revealed complete conversion of one regioisomer. This was followed by elevating the internal temperature to 130° C. and further stirring for 2 hours at this temperature level until HPLC monitoring indicated complete conversion of the other regioisomer. Afterwards the reaction solution was cooled down to 22° C. and washed with 2×100 mL deionized water. The aqueous phase was extracted with 2×50 mL of chlorobenzene. The combined organic phases were then dried over MgSO₄, the drying agent was filtered off and the filtrate was concentrated to dryness at 60° C. and down to 13 mbar to leave 20.80 g (66.1% purity, 78.45 mmol, 40% yield) of rac-2-(1,3-dimethylbut-2-enyl)aniline as a clear, red oil. ¹H-NMR (600 MHz; CDCl₃) δ=7.17-7.14 (m, 1H), 7.05-7.01 (m, 1H), 6.79-6.75 (m, 1H), 6.66 (dd, J=6.0 Hz, 12.0 Hz, 1H), 5.11 (d, J=6.0 Hz, 1H), 3.64-3.54 (m, 3H), 1.75 (s, 3H), 1.71 (s, 3H), 1.33 (d, J=9.0 Hz, 3H).

### Example 6

### Preparation of rac-2-(1,3-dimethylbut-2-enyl)-4-fluoroaniline (a Compound According to Formula (I)) Via a Telescoped Reaction

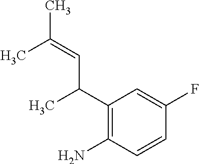

In a 100 mL four-necked reaction flask equipped with a thermometer was placed 20.00 g (98.5% purity, 196.69 mmol, 1.00 eq) of rac-4-methylpent-3-en-2-ol and 50 mL of chlorobenzene. The solution was cooled to 0° C. and inertized via Argon. To the solution was dosed 17.75 g (99% purity, 64.91 mmol, 0.33 eq) of phosphorus tribromide at 0-4° C. over a period of 1 hour. After 30 minutes of post-stirring at 0° C. the reaction mixture was allowed to warm up to 22° C. and was subsequently transferred into a dropping funnel. Of the two separated phases the dark viscous lower layer was discarded. A second 100 mL four-necked reaction flask was equipped with a thermometer and with the dropping funnel containing the product phase of the first reaction. Then 20.97 g (99% purity, 186.85 mmol, 0.95 eq) of aniline and 19.09 g (99% purity, 186.85 mmol, 1.00 eq) of N-methylmorpholine were dissolved in 35 mL of chlorobenzene at 22° C. To this solution was dosed the product solution of the first reaction within 4 hours maintaining an internal temperature of 22-24° C. via waterbath cooling. After 1 hour of post-stirring at 22° C. the organic phase was washed with 2×200 mL deionized water and was subsequently degassed with Argon for 1 hour. To the organic phase was then added 1.23 g (99% purity, 6.39 mmol, 3.3 mol %) of 4-toluenesulfonic acid monohydrate in one portion at 22° C. Afterwards the reaction mixture was heated to 95° C. internal temperature and stirred for 6 hours until HPLC monitoring revealed complete conversion of one regioisomer. This was followed by elevating the internal temperature to 130° C. and further stirring for 2 hours at this temperature level until HPLC monitoring indicated complete conversion of the other regioisomer. Afterwards the reaction solution was cooled down to 22° C. and washed with 2×100 mL deionized water. The aqueous phase was extracted with 2×50 mL of chlorobenzene. The combined organic phases were then dried over MgSO₄, the drying agent was filtered off and the filtrate was concentrated to dryness at 40° C. and down to 10 mbar to leave 18.10 g (50.8% purity, 47.60 mmol, 24% yield) of rac-2-(1,3-dimethylbut-2-enyl)-4-fluoroaniline as a clear, red oil. ¹H-NMR (600 MHz; CDCl₃) δ=6.78-6.70 (m, 1H), 6.63-6.56 (m, 2H), 5.05 (d, J=6.0 Hz, 1H), 3.61-3.56 (m, 1H), 3.43 (bs, 2H), 1.72 (s, 3H), 1.69 (s, 3H), 1.27 (d, J=9.0 Hz, 3H).

### Example 7

### Preparation of rac-1,1-dimethyl-3-propyl-indan-4-amine Using Aqueous Sulfuric Acid

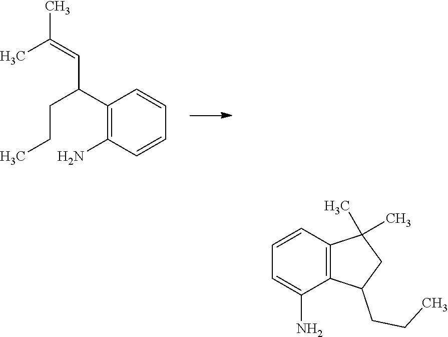

To a 1000 mL four-necked round-bottomed flask equipped with a thermometer, a mechanical stirrer and two dropping funnels was dosed a mixture comprising 350.0 g of recycled sulfuric acid (90% purity) and 44.0 g fresh sulfuric acid (90% purity) simultaneously with 100.0 g (74% purity, 0.36 mol, 1.0 eq) of rac-1,1-dimethyl-3-propyl-indan-4-amine at 0-10° C. internal temperature over a period of 3 hours under vigorous stirring. Some jelly-like solids were formed intermediary, which dissolved completely during the end of the addition. After completed dosing, the dark red reaction solution was added onto 800.0 g of deionized icy water under vigorous stirring. The solid was filtered off and washed with a total of 400 mL of deionized water. The combined filtrate was subjected to distillation at 20 mbar and 150° C. in order to concentrate the sulfuric acid back to 90% purity. The solid was suspended in 500 mL of deionized water and 150 mL of methylcyclohexane. To this suspension was added 86.8 g (1.08 mol, 3.0 eq) of 50 w % soda lye. Two liquid phases were formed, of which the lower phase was separated. The aqueous phase was extracted once with another 150 mL of methylcyclohexane. The combined organic phases were then washed with 100 mL of saturated brine. After phase separation the organic phase was concentrated via distillation at 40° C. down to a vacuum of 25 mbar to leave 88.4 g (67% purity, 0.29 mol, 81% yield) of rac-1,1-dimethyl-3-propyl-indan-4-amine as a dark red oil. ¹H-NMR (600 MHz; CDCl₃) δ=7.02 (t, J=7.5 Hz, 1H), 6.59 (d, J=7.5 Hz, 1H), 6.47 (d, J=7.5 Hz, 1H), 3.56 (bs, 2H), 3.11-3.06 (m, 1H), 2.09 (dd, J=12.0 Hz, 24.0 Hz, 1H), 1.92-1.86 (m, 2H), 1.76 (dd, J=6.0 Hz, 12.0 Hz, 1H), 1.55-1.32 (m, 2H), 1.30 (s, 3H), 1.21 (s, 3H), 0.97 (t, J=8.0 Hz, 3H).

### Example 8

### Preparation of rac-1,1-dimethyl-3-propyl-indan-4-amine Using Anhydrous HF

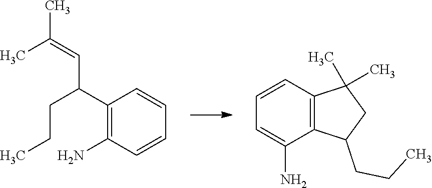

In a 30 mL Nalgene® laboratory bottle were placed 10.0 g (10 mL) of anhydrous hydrogen fluoride (b.p. 19.5° C., m.p. −83.6° C.). The content was cooled to −30° C. and 5.0 g (71% purity, 17.39 mmol, 1.0 eq) of rac-1,1-dimethyl-3-propyl-indan-4-amine was added to the reaction mixture in small portions. The bottle was sealed by a stopper and the reaction mixture was allowed to warm to 25° C. Afterwards stirring was prolonged for 24 hours under the same conditions. The content of the bottle was then poured into a 250 mL plastic beaker, and excess of hydrogen fluoride was evaporated at open air within the fumehood. The oily residue was treated with 20 mL of 10 w % aqueous solution of sodium bicarbonate until pH 7 was obtained (ceasing CO₂ gas formation) and extracted with 2×50 mL of dichloromethane. The combined dichloromethane extracts were then washed with 30 mL of concentrated brine, dried over sodium sulfate and evaporated via distillation to leave 4.88 g (62% purity, 14.88 mmol, 86% yield) of rac-1,1-dimethyl-3-propyl-indan-4-amine as a dark red oil. ¹H-NMR (600 MHz; CDCl₃) δ=7.02 (t, J=7.5 Hz, 1H), 6.59 (d, J=7.5 Hz, 1H), 6.47 (d, J=7.5 Hz, 1H), 3.56 (bs, 2H), 3.11-3.06 (m, 1H), 2.09 (dd, J=12.0 Hz, 24.0 Hz, 1H), 1.92-1.86 (m, 2H), 1.76 (dd, J=6.0 Hz, 12.0 Hz, 1H), 1.55-1.32 (m, 2H), 1.30 (s, 3H), 1.21 (s, 3H), 0.97 (t, J=8.0 Hz, 3H).

### Example 9

### Preparation of rac-1,1,3-trimethyl-indan-4-amine Using Aqueous Sulfuric Acid

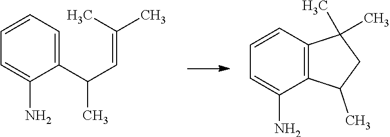

To a 100 mL four-necked round-bottomed flask equipped with a thermometer was added 91.00 g (90% purity, 835.05 mmol, 10.66 eq) of aqueous sulfuric acid. To the acid were then dosed 20.80 g (66% purity, 78.32 mmol, 1.0 eq) of rac-2-(1,3-dimethylbut-2-enyl)aniline at 5-10° C. internal temperature over a period of 1 hour under vigorous stirring. Some jelly-like solids were formed intermediary, which dissolved completely during the end of the addition. The reaction mixture was stirred for further 3 hours at 22° C. until HPLC monitoring indicated complete conversion. Afterwards the reaction solution was added onto 160.0 g of deionized icy water under vigorous stirring. The resulting mixture was then completely neutralized until pH 10 via addition of aqueous sodium hydroxide (20 w %). The resulting solid material was filtered off and was discarded. The filtrate was extracted with 2×100 mL t-butylmethylether. The combined organic phases were then dried over MgSO₄. The drying agent was filtered off and the organic phase was concentrated via distillation at 40° C. down to a vacuum of 10 mbar to leave 17.10 g (45% purity, 43.86 mmol, 56% yield) of rac-1,1,3-trimethylindan-4-amine as a red oil. ¹H-NMR (600 MHz; CDCl₃) δ=7.03 (t, J=7.0 Hz, 1H), 6.60 (d, J=7.0 Hz, 1H), 6.50 (d, J=7.0 Hz, 1H), 3.59 (bs, 2H), 3.23-3.21 (m, 1H), 2.21 (dd, J=8.0 Hz, 12.0 Hz, 1H), 1.62 (dd, J=8.0 Hz, 12.0 Hz, 1H), 1.34 (d, J=8.0 Hz, 3H), 1.31 (s, 3H), 1.23 (s, 3H).

### Example 10

### Preparation of rac-7-fluoro-1,1,3-trimethyl-indan-4-amine Using Aqueous Sulfuric Acid

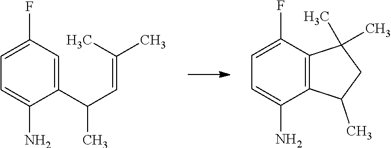

To a 250 mL four-necked round-bottomed flask equipped with a thermometer was added 226.32 g (90% purity, 2238.33 mmol, 47.05 eq) of aqueous sulfuric acid. To the acid were then dosed 18.10 g (51% purity, 47.58 mmol, 1.00 eq) of rac-2-(1,3-dimethylbut-2-enyl)-4-fluoroaniline at 5-10° C. internal temperature over a period of 1 hour under vigorous stirring. Some jelly-like solids were formed intermediary, which dissolved completely during the end of the addition. The reaction mixture was stirred for 1 hour at 15° C. until HPLC monitoring indicated complete conversion. Afterwards the reaction solution was added onto 150.0 g of deionized icy water under vigorous stirring. The resulting suspension was filtered and the filtrate was discarded. The solid was re-suspended in 100 mL of deionized water and the resulting mixture was neutralized with 25 mL of aqueous sodium hydroxide (20 w %). The resulting suspension was again filtered. The solid was then washed with 1×25 mL deionized water. After drying at 40° C. and 70 mbar 8.00 g (67% purity, 27.60 mmol, 58% yield) of rac-7-fluoro-1,1,3-trimethyl-indan-4-amine were obtained as an off-white solid. ¹H-NMR (600 MHz; CDCl₃) δ=6.68-6.65 (m, 1H), 6.44-6.41 (m, 1H), 3.25-3.16 (m, 3H), 2.22 (dd, J=8.0 Hz, 12.0 Hz, 1H), 1.65 (dd, J=8.0 Hz, 12.0 Hz, 1H), 1.43 (s, 3H), 1.35 (s, 3H), 1.32 (d, J=8.0 Hz, 3H).

1. Process for preparation of a compound of the formula (V) 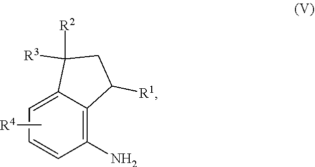
in which
R¹ represents (C₁-C₄)alkyl;
R² represents hydrogen or (C₁-C₈)alkyl;
R³ represents hydrogen or (C₁-C₈)alkyl, provided that R² and R³ are not hydrogen at the same time;
R⁴ represents hydrogen, halogen, (C₁-C₄)alkyl or (C₁-C₄)haloalkyl, comprising (d) reacting a compound of Formula (I) 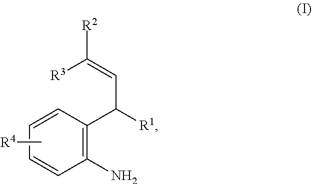
wherein the definitions of the substituents R¹, R², R³ and R⁴ listed in the formula (I) are the same as in the formula (V), is reacted with aqueous sulfuric acid or anhydrous HF.

2. The process for the preparation of a compound of formula(V) according to claim 1 comprising (b), (c) and (d),
wherein in (b) a compound of formula (III) 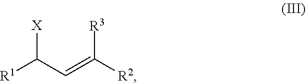
in which X represents a halogen or O—SO₂R wherein R is a methyl, phenyl or tolyl group
is reacted with a compound of formula (IIIa) 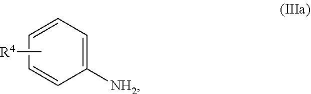
to obtain a compound of formula (IV) 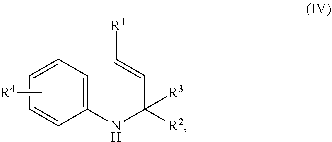
and wherein in (c) a compound of formula (IV), is rearranged in the presence of an acid to obtain a compound of formula (I) 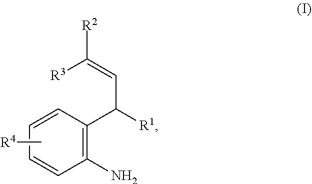
and wherein in (d) a compound of formula (I) is reacted with aqueous sulfuric acid or anhydrous hydrogen fluoride (HF), to yield a compound of formula (V).

3. The process according to claim 1, wherein R¹ is n-propyl, R² and R³ are methyl and R⁴ is hydrogen.

4. The process according to claim 1, wherein R¹, R² and R³ are methyl and R⁴ is hydrogen.

5. The process according to claim 1, wherein in (a) a compound of formula (II) 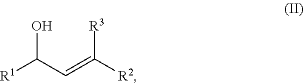
is converted in the presence of an activating agent to a compound of formula (III).

6. The process according to claim 1, wherein a solvent in (a) and/or (b) and/or (c) is selected from the group consisting of chlorobenzene, toluene, xylene, anisole and trifluorobenzene, optionally the solvent is chlorobenzene.

7. The process according to claim 1, wherein (a) is carried out at a temperature in a range of from −5° C. to 120° C.; and/or (b) is carried out at a temperature in a range of from 10° C. to 90° C.; and/or (c) is carried out at a temperature in a range of from 80° C. to 140° C.

8. The process according to claim 1, wherein (b) is conducted in the presence of an additional base, wherein the additional base is selected from the group consisting of N-methylmorpholine, diisopropylethylamine, N,N,N′,N′-tetramethylguanidine, tri-n-butylamine, triethylamine, 1,4-Diazabicyclo[2.2.2]octane, 1,8-diazabicyclo[5.4.0]-undec-7-ene, N-Methylimidazole, potassium tert-butoxide, sodium tert-butoxide and lithium tert-butoxide, optionally the base used in (b) is selected from the group consisting of N-methylmorpholine, diisopropylethylamine, N,N,N′,N′-tetramethylguanidine, tri-n-butylamine and triethylamine, optionally the base used in (b) is selected from the group consisting of N-methylmorpholine and diisopropylethylamine.

9. The process according to claim 1, wherein the acid in (c) is selected from the group consisting of 4-toluenesulfonic acid, methanesulfonic acid, trifluoromethanesulfonic acid, Zn(OTf)₂, Cu(OTf)₂, Ni(OTf)₂, Fe(OTf)₂, Fe(OTf)₃, benzenesulfonic acid, sulfuric acid, sulfamic acid, phenylphosphonic acid, ethylphosphonic acid, phosphoric acid, acetic acid, chloroacetic acid, dichloroacetic acid, trichloroacetic acid and trifluoroacetic acid, optionally the acid in (c) is methanesulfonic acid, optionally the acid in (c) is 4-toluenesulfonic acid.

10. The process according to claim 1, wherein (d) is carried out at a temperature in a range of from −80° C. to 30° C., optionally at a temperature in a range of from −50° C. to 20° C.; optionally at a temperature in a range of from −30° C. to 20° C.

11. The process according to claim 1, wherein in (d) the aqueous sulfuric acid having a concentration of at least 85 w % is used.

12. The process according to claim 1, wherein an amount of used aqueous sulfuric acid or anhydrous HF is in a range of from 3-45 molar equivalents, optionally of from 6 to 40 molar equivalents, optionally of from 9 to 35 molar equivalents based on the total amount of the compound of the formula (II).

13. A process for preparation of a compound of the formula (VII) 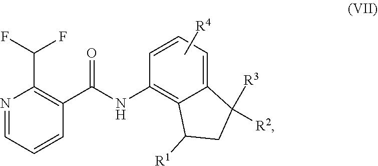
wherein in formula (VII),
comprising the process according to claim 1 and further comprising (e), wherein a compound of the formula (V) is reacted with a compound of formula (VI) 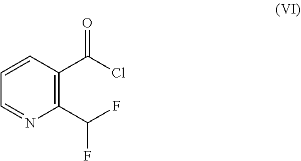 [SMILES: O=C(Cl)c1cccnc1C(F)F]
to obtain a compound of formula (VII).

14. Compound of formula (III) 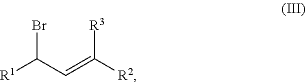
wherein
R¹ represents (C₁-C₄)alkyl;
R² represents hydrogen or (C₁-C₈)alkyl;
R³ represents hydrogen or (C₁-C₈)alkyl, provided that R² and R³ are not hydrogen at the same time;
R⁴ represents hydrogen, halogen, (C₁-C₄)alkyl or (C₁-C₄)haloalkyl.

15. Compound of formula (IV) 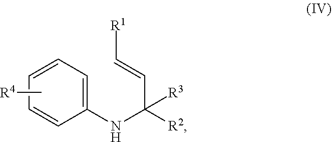
Wherein
R¹ represents (C₁-C₄)alkyl;
R² represents hydrogen or (C₁-C₈)alkyl;
R³ represents hydrogen or (C₁-C₈)alkyl, provided that R² and R³ are not hydrogen at the same time;
and
R⁴ represents hydrogen.
